(19) United States
(12) Patent Application Publication
(10) Pub. No.: US 2014/0170133 A1
(43) Pub. Date: Jun. 19, 2014

(54) ALTERATION OF PROTEOLYTIC CLEAVAGE OF BOTULINUM NEUROTOXINS

(71) Applicant: Jurgen Frevert, Berlin (DE)

(72) Inventor: Jurgen Frevert, Berlin (DE)

(21) Appl. No.: 14/236,454

(22) PCT Filed: Jul. 26, 2012
(86) PCT No.: PCT/EP2012/064690

§ 371 Date: Jan. 31, 2014

Related U.S. Application Data
(60) Provisional application No. 61/574,596, filed on Aug. 4, 2011.

(30) Foreign Application Priority Data
Aug. 4, 2011 (EP) 11176531.9

(51) Int. Cl. A61K 38/48; C12N 15/52

(52) U.S. Cl. CPC A61K 38/4893; C12N 15/52
USPC 424/94.67; 536/23.2; 435/212; 514/44 R; 435/320.1; 435/252.33

(57) ABSTRACT

The present invention pertains to a polynucleotide encoding a modified neurotoxin polypeptide comprising a modified neurotoxin light chain and a heavy chain, said modified light chain having at least one modification conferring altered cleavage by calpain proteases. Further encompassed by the present invention are vectors and host cells comprising the polynucleotide of the invention as well as polypeptides encoded by the said polynucleotide. In addition, the invention relates to compositions comprising the polynucleotide, vector, host cell or polypeptide of the invention as a medicament.

The present invention relates to a polynucleotide encoding a modified neurotoxin polypeptide comprising a modified neurotoxin light chain and a heavy chain, said modified light chain having at least one modification conferring altered cleavage by calpain proteases. Further encompassed by the present invention are vectors and host cells comprising the polynucleotide of the invention as well as polypeptides encoded by the said polynucleotide. In addition, the invention relates to compositions comprising the polynucleotide, vector, host cell or polypeptide of the invention as a medicament.

Clostridium botulinum and Clostridium tetani produce highly potent neurotoxins, i.e. botulinum toxins (BoNTs) and tetanus toxin (TeNT), respectively. These Clostridial neurotoxins specifically bind to neuronal cells and disrupt neurotransmitter release. Each toxin is synthesized as an inactive unprocessed approximately 150 kDa single-chain protein. The posttranslational processing involves formation of disulfide bridges, and limited proteolysis (nicking) by bacterial protease(s). Active di-chain neurotoxin consists of two chains, an N-terminal light chain of approx. 50 kDa and a heavy chain of approx. 100 kDa linked by a disulfide bond. Neurotoxins structurally consist of three domains, i.e. the catalytic light chain, the heavy chain encompassing the translocation domain (N-terminal half) and the receptor binding domain (C-terminal half), see Krieglstein 1990, Eur Biochem 188, 39; Krieglstein 1991, Eur J Biochem 202, 41; Krieglstein 1994, J Protein Chem 13, 49.

Clostridium botulinum secretes seven antigenically distinct serotypes designated A to G of the BoNTs. All serotypes together with the related TeNT secreted by Clostridium tetani, are zinc (Zn²⁺)-dependent endoproteases that block synaptic exocytosis by cleaving SNARE proteins and, in particular, SNAP-25, which is cleaved by BoNT/A, BoNT/C1 and BoNT/E. BoNTs cause, inter alia, the flaccid muscular paralysis seen in botulism and tetanus, see Fischer 2007, PNAS 104, 10447.

Despite its toxic effects, BoNTs have been used as therapeutic agents in a large number of diseases. BoNT serotype A (BoNT/A) was approved for human use in the United States in 1989 for the treatment of strabism, blepharospasm, and other disorders. It is commercially available as a protein preparation, for example, under the tradename BOTOX (Allergan Inc.) or under the tradename DYSPORT (Ipsen Ltd.). In these preparations, the neurotoxin is integrated in a protein complex with so called complexing proteins. For therapeutic application, the complex is injected directly into the muscle to be treated. At physiological pH, the toxin is released from the protein complex to exert the desired pharmacological effect. An improved BoNT/A preparation being free of complexing proteins is available under the tradename XEOMIN (Merz Pharmaceuticals GmbH).

BoNTs, in principle, weaken voluntary muscle strength and are, therefore, effective therapeutic agents for the therapy of diseases such as strabism, focal dystonia, including cervical dystonia, and benign essential blepharospasm. They have been further shown to relief hemifacial spasm, and focal spasticity, and moreover, to be effective in a wide range of other indications, such as gastrointestinal disorders, hyperhidrosis, and cosmetic wrinkle correction, see Jost 2007, Drugs 67, 669.

However, the effect of BoNTs is only temporary, which is the reason why repeated administration of BoNTs may be required to maintain a therapeutic effect. Moreover, the more frequently these drugs are applied the higher will be the risk for an adverse immune response against the neurotoxin applied. Further, some patients develop anti-neurotoxin antibodies and, thereby, become non-responders to therapies by conventional BoNTs. BoNTs are in some indications applied only locally. However, their diffusion potential makes a controlled local application difficult. In general, the production of these highly toxic polypeptides is cumbersome and needs special care with respect to safety issues and thus is expensive. In light of these drawbacks of conventional neurotoxin polypeptides as drugs, means for controlling and/or improving the biological activity of neurotoxins would be highly appreciated for the neurotoxin therapy.

Thus, the technical problem underlying the present invention could be seen as the provision of means and methods which comply with the aforementioned needs. This technical problem has been solved by the embodiments characterized in the claims and herein below.

The present invention relates to a polynucleotide encoding a modified neurotoxin polypeptide comprising a modified neurotoxin light chain and a heavy chain, said modified light chain having at least one modification conferring altered cleavage by calpain proteases.

The term “neurotoxin” as used herein means a Clostridial molecule which is capable of interfering with the functions of a cell, including a neuron. Preferably, the neurotoxin is a polynucleotide encoding the neurotoxin polypeptide or a neurotoxin polypeptide. The interfered cell function can be exocytosis. The neurotoxin can be naturally occurring or recombinant. Active di-chain neurotoxin polypeptide consists of two chains, an N-terminal light chain of approx. 50 kDa and a heavy chain of approx. 100 kDa linked by a disulfide bond. Neurotoxins structurally consist of three domains, i.e. the catalytic light chain, the heavy chain encompassing the translocation domain (N-terminal half) and the receptor binding domain (C-terminal half).

The term “modified neurotoxin” as used herein means a Clostridial neurotoxin which includes a modification. Preferably, the modification is within the neurotoxin light chain.

The term “light chain” as used herein means the light chain of a Clostridial neurotoxin. It has a molecular weight of about 50 kDa, and can be referred to as light chain or as the proteolytic domain of a Clostridial neurotoxin. The light chain is believed to be effective as an inhibitor of exocytosis, including as an inhibitor of neurotransmitter, e.g. acetylcholine, release when the light chain is present in the cytoplasm of a target cell, such as a neuron.

The term “modified light chain” as used herein denotes the light chain of a Clostridial neurotoxin which includes a modification. Preferably, the modification is a structural modification. The modified neurotoxin light chain is structurally different from a naturally occurring neurotoxin light chain, i.e. a non-modified neurotoxin light chain. This structural modification in the light chain of the neurotoxin changes (i) the half-life (time), (ii) the biological activity, (iii) the biological persistence, and/or (iv) the immunogenicity of the neurotoxin in an organism, relative to the neurotoxin from which the modified light chain is derived, i.e. a non-modified neurotoxin.

The term “heavy chain” as used herein refers to the heavy chain of a Clostridial neurotoxin. It has a molecular weight of about 100 kDa.

The term “polynucleotide” as used herein refers to single- or double-stranded DNA molecules as well as to RNA molecules. Encompassed by the said term is genomic DNA, cDNA, hnRNA, mRNA as well as all naturally occurring or artificially modified derivatives of such molecular species. The polynucleotide may be in an aspect a linear or circular molecule. Moreover, in addition to the nucleic acid sequences encoding the aforementioned modified neurotoxin polypeptide, a polynucleotide of the present invention may comprise additional sequences required for proper transcription and/or translation such as 5′- or 3′-UTR sequences. The polynucleotide of the present invention encodes a modified neurotoxin polypeptide as described in more detail herein. The modified neurotoxin polypeptide and, in particular, its modified light chain and heavy chain are derivable from one of the antigenically different serotypes of Botulinum Neurotoxins (BoNT), i.e. BoNT/A, BoNT/B, BoNT/C1, BoNT/D, BoNT/E, BoNT/F, BoNT/G, or Tetanus Neurotoxin (TeNT). Neurotoxin polypeptides comprise an N-terminal light chain of approximately 50 kDa, and a C-terminal heavy chain of approximately 100 kDa, linked by a disulfide bond. The neurotoxins are translated as single chain precursor molecules and become proteolytically cleaved into a mature, biologically active di-chain form during processing. The neurotoxin polypeptide (prior to the modification of the invention) comprises the light and heavy chain of neurotoxin BoNT/A, BoNT/B, BoNT/C1, BoNT/D, BoNT/E, BoNT/F, BoNT/G or TeNT. Said light and heavy chain of the neurotoxin polypeptide (prior to the modification) comprise an amino acid sequence as shown in any one of SEQ ID NO: 1 (BoNT/A), SEQ ID NO: 2 (BoNT/C1), or SEQ ID NO: 3 (BoNT/E) or in Swiss-Prot: B1INP5.1 (BoNT/B). The amino acid sequence of BoNT/A (comprising the heavy chain and light chain) is, for example, shown in GenBank accession number YP_{—}001253342.1. The amino acid sequence of BoNT/C1 (comprising the heavy chain and light chain) is, for example, shown in Swiss-Prot. accession number P18640.2. The amino acid sequence of BoNT/E (comprising the heavy chain and light chain) is, for example, shown in GenBank accession number CAA44558.1. In one aspect, the structural modification in the BoNT/A light chain comprises one or more mutations selected from the group consisting of a (i) E126A mutation (glutamic acid at position 126 of the light chain is replaced by an alanine), a (ii) L127A mutation (leucine at position 127 is replaced by an alanine), a (iii) F213I mutation (phenylalanine at position 213 of the light chain is replaced by an isoleucine) or an (iv) A214I mutation (alanine at position 214 of the light chain is replaced by an isoleucine), with the amino acid sequence numbering as in GenBank accession number YP_{—}001253342.1 or in SEQ ID NO: 1. In another aspect, the structural modification in the BoNT/A light chain comprises one or more mutations selected from the group consisting of a (i) E126D mutation (glutamic acid at position 126 of the light chain is replaced by an aspartic acid), a (ii) L127V mutation (leucine at position 127 is replaced by an valine), a (iii) F213Y mutation (phenylalanine at position 213 of the light chain is replaced by an tyrosine) or an (iv) A214G mutation (alanine at position 214 of the light chain is replaced by an glycine), with the amino acid sequence numbering as in GenBank accession number YP_{—}001253342.1 or in SEQ ID NO: 1. In yet another aspect, the structural modification in the BoNT/A light chain comprises one or more mutations selected from the group consisting of a (i) E126A or D mutation, a (ii) L127A or V mutation, a (iii) F213I or Y mutation or an (iv) A214I or G mutation, with the amino acid sequence numbering as in GenBank accession number YP_{—}001253342.1 or in SEQ ID NO: 1. Preferably, said one or more mutations is/are within the BoNT/A light chain. More preferably, the BoNT/A light chain comprises an E126A mutation, a L127A mutation, a F213I mutation and an A214I mutation, with the amino acid sequence numbering as in GenBank accession number YP_{—}001253342.1 or in SEQ ID NO: 1. In another aspect, the BoNT/A light chain comprises an E126D mutation, L127V mutation, F213Y mutation, and A214G mutation, with the amino acid sequence numbering as in GenBank accession number YP_{—}001253342.1 or in SEQ ID NO 1. However, it is encompassed by the scope of the present invention that other and/or further mutations can be introduced into the light chain, either in addition to one or more of the mutations mentioned above or instead of these mutations. Preferably, said mutation(s) is/are within (a) calpain protease cleavage site(s).

The term “calpain proteases” or “calpains” as used herein refers to proteins belonging to the family of calcium-dependent, non-lysosomal cysteine proteases, i.e. proteolytic enzymes expressed ubiquitously in mammals and many other organisms. Calpains constitute the C2 family of protease clan CA in the MEROPS database. The calpain proteolytic system includes the calpain proteases, the small regulatory subunit CAPNS1, and the endogenous calpain-specific inhibitor, calpastatin.

The calpain protease family contains 14 members with μ-calpain (calpain-1) and m-calpain (calpain-2) being the most well-characterized. The accession number of μ-calpain (calpain-1, catalytic subunit, isoform a) is shown in NP001185798.1, whereas the accession number of m-calpain (calpain-2, catalytic subunit, isoform 1) is depicted in NP001739.2. Structurally, the calpains contain two subunits; an 80 kDa catalytic subunit and a 28 kDa regulatory subunit that functions as a chaperone to stabilize the 80 kDa structure. Calpains are regulated by Ca²⁺ concentration, phosphorylation, calpastatin and probably by altering their subcellular localization, e.g. by limiting access to the substrate. These endopeptidases have numerous biological functions including, but not limited to, remodeling of cytoskeletal attachments to the plasma membrane during cell fusion and cell motility, proteolytic modification of molecules in signal transduction pathways, degradation of enzymes controlling progression through the cell cycle, regulation of gene expression, substrate degradation in some apoptotic pathways, and an involvement in long-term potentiation (Chowdhury et al. 2008, Comp Biochem Physiol B Biochem Mol. Biol. 151, 10). μ-calpain (calpain-1) and m-calpain (calpain-2) have also been found to be involved in the degradation of botulinum neurotoxins in neurons, which means that botulinum neurotoxins are recognized and cleaved by calpains. Amongst protein substrates, primary amino acid sequences and tertiary structure elements are likely responsible for directing cleavage to a specific substrate; see e.g. Tompa 2004, J Biol Chem 279, 20775 or Cuerrier 2005, J Biol Chem 280, 40632.

The term “modified light chain having at least one modification conferring altered cleavage by calpain proteases” as used herein denotes a modification within the neurotoxin light chain which results in (i) increased, (ii) decreased, or (iii) no cleavage, by calpains of the modified neurotoxin light chain.

The term “modification” means any change to a neurotoxin light chain which makes it physically or chemically different from a neurotoxin light chain without the structural modification, e.g. a naturally occurring neurotoxin light chain. Preferably, the modification is a structural modification. The structural modification can comprise e.g. the introduction of one or more exogenous or endogenous calpain recognition and/or cleavage site(s) into the neurotoxin light chain. Proteolytic cleavage by the calpains of a neurotoxin light chain includes recognition and cleavage of the neurotoxin light chain by the calpains. Recognition of the neurotoxin light chain occurs at the calpain recognition site within the neurotoxin light chain. Proteolytic cleavage occurs at the calpain cleavage site within the neurotoxin light chain. Recognition and proteolysis by calpains of the neurotoxin light chain results in the degradation of the neurotoxin light chain, thereby inactivating the proteolytic activity. For example, it has been found that μ-calpain (calpain-1) and m-calpain (calpain-2) are involved in the degradation processes of neurotoxins.

In light of the above, the term “calpain recognition site” as used herein refers to a site, e.g. a primary amino acid sequence or tertiary structure elements, on the neurotoxin light chain recognized by the calpain proteases. The term “calpain cleavage site” as used herein refers to the cleavage site within the neurotoxin light chain cleaved by a calpain protease.

The recognition site and cleavage site of the calpain proteases in the neurotoxin light chain can be identical or they can differ from each other; see, e.g. Tompa 2004, J Biol Chem 279, 20775.

The term “endogenous calpain recognition and/or cleavage site” as used herein means a calpain recognition and/or cleavage site naturally occurring in the neurotoxin light chain. The term “exogenous calpain recognition and/or cleavage site” as used herein means a calpain recognition and/or cleavage site which does not naturally occur in the neurotoxin light chain, e.g. a heterologous calpain recognition and/or cleavage site derived from a different organism or a recombinant calpain recognition and/or cleavage site. The structural modification can also be a mutation within an exogenous or endogenous calpain recognition and/or cleavage site in the neurotoxin light chain. A mutation in a nucleic acid sequence as used herein can be a deletion, addition or substitution of one or more nucleotides in a DNA sequence coding for a calpain recognition and/or cleavage site. A mutation in a protein sequence can be a deletion, addition or substitution of one or more amino acid residues in a protein sequence of the calpain recognition and/or cleavage site. Such a mutation in a protein sequence of the calpain recognition and/or cleavage site can be, for example, a substitution of one or more amino acid residue(s) at positions P1, P2, P3, P4, P5, P1′, P2′, and/or P3′ position of the calpain recognition and/or cleavage site in the neurotoxin light chain. Preferably, the protein sequence of the calpain recognition and/or cleavage site within the light chain of BoNT/A comprises, for example, Gly-Lys-Phe-Ala-Thr-Asp-Pro (GKFATDP) (SEQ ID NO: 4), with the Glycine corresponding to amino acid residue 211 of the BoNT/A light chain sequence as shown, e.g. in GenBank number YP_{—}001253342.1 or SEQ ID NO: 1. More specifically, Gly corresponds to the P3 position, Lys to the P2 position, Phe to the P1 position, Ala to the P1′ position, Thr to the P2′ position, Asp to the P3′ position and Pro to the P4′ position. In another aspect, the protein sequence of the calpain recognition and/or cleavage site within the light chain of BoNT/A comprises Glu-Leu-Lys-Val-11e-Asp (ELKVID) (SEQ ID NO: 5), with the Glu corresponding to amino acid residue 126 of the BoNT/A light chain sequence as shown, e.g. in GenBank number YP_{—}001253342.1 or SEQ ID NO: 1. More specifically, Glu corresponds to the P3 position, Leu to the P2 position, Lys to the P1 position, Val to the P1′ position, Be to the P2′ position and Asp to the P3′ position. In other aspects, the calpain recognition and/or cleavage site for calpain-1 within the light chain of BoNT/A comprises Glu-Asp-Thr-Ser-Gly-Lys (SEQ ID NO: 6), Gly-Leu-Glu-Val-Ser-Phe (SEQ ID NO: 7), Leu-Asn-Lys-Ala-Lys-Ser (SEQ ID NO: 8), Val-Asp-Lys-Leu-Lys-Phe (SEQ ID NO: 9), or Val-Leu-Asn-Arg-Lys-Thr (SEQ ID NO: 10), wherein the BoNT/A light chain sequence is as shown, e.g. in GenBank number YP_{—}001253342.1 or SEQ ID NO: 1. In other aspects, the calpain recognition and/or cleavage site for calpain-2 within the light chain of BoNT/A comprises Ile-Val-Gly-Thr-Thr-Ala (SEQ ID NO: 11), or Gly-Thr-Thr-Ala-Ser-Leu (SEQ ID NO: 12), wherein the BoNT/A light chain sequence is as shown, e.g. in GenBank number YP_{—}001253342.1 or SEQ ID NO: 1. One, two, three, four, five, or even all of the mentioned amino acid residues can be replaced by another amino acid residue. A preferred structural modification within the calpain recognition and/or cleavage site Gly-Lys-Phe-Ala-Thr-Asp-Pro (GKFATDP) (SEQ ID NO: 4) of the light chain of BoNT/A comprises an F213I or F213Y mutation (phenylalanine at position 213 of the light chain is replaced by an isoleucine or tyrosine) and/or an A214I or A214G mutation (alanine at position 214 of the light chain is replaced by and isoleucine or glycine), with the BoNT/A light chain sequence as indicated above. A preferred structural modification within the calpain recognition and/or cleavage site Glu-Leu-Lys-Val-11e-Asp (ELKVID) (SEQ ID NO: 5) comprises an E126A or E126D mutation (glutamic acid at position 126 of the light chain or the P3 position is replaced by an alanine or aspartic acid) and/or a L127A or L1271 mutation (leucine at position 127 or P2 position is replaced by an alanine or isoleucine), with the BoNT/A light chain sequence as indicated above. Mutations to a protein sequence can be the result of mutations to DNA sequences that when transcribed and the resulting mRNA translated produce the mutated protein sequence. The term “having at least one modification” as used herein means that the modified neurotoxin light chain has one, two, three or even more modifications conferring altered cleavage by calpain proteases. “Altered cleavage” as used herein means increased cleavage or decreased cleavage or no cleavage of the modified neurotoxin light chain by calpains, in comparison to a naturally occurring neurotoxin light chain, i.e. a non-modified light chain. Said modification in the neurotoxin light chain alters advantageously the biological persistence, biological half life, biological activity and/or immunogenicity of a thus modified neurotoxin, preferably the duration of the biological activity of the modified neurotoxin.

The term “biological activity” or “activity” of a non-modified or modified neurotoxin as used herein denotes the amount of cellular exocytosis inhibited from a cell per unit of time, such as exocytosis of a neurotransmitter, e.g. acetylcholine, from a target cell, such as a neuron. More specifically, it refers to the biological activity of a mature (non-modified or modified) di-chain neurotoxin polypeptide exhibiting a) receptor binding, b) internalization, c) translocation across the endosomal membrane into the cytosol, and/or d) endoproteolytic cleavage of proteins involved in synaptic vesicle fusion. The term “duration of biological activity” as used herein means the time period of the biological activity of a neurotoxin which can be influenced, i.e. altered, by the modification of the neurotoxin light chain, as described herein.

The term “biological persistence” or “persistence” as used herein means the time of duration of interference or influence caused by a non-modified neurotoxin or a modified neurotoxin with a cellular, such as a neuronal, function, including the temporal duration of an inhibition of exocytosis, such as exocytosis of neurotransmitter (e.g. acetylcholine) from a cell, such as a neuron.

The term “biological half life (time)” or “half life (time)” as used herein means the time that the concentration of a non-modified neurotoxin or a modified neurotoxin is reduced to half of the original concentration in a mammalian cell, such as in a mammalian neuron. Preferably, the mammalian neuron is a human neuron.

The term “immunogenicity” as used herein means the ability of a particular substance, such as an antigen (, e.g. a neurotoxin,) or epitope, to provoke an immune response in the body of a human or animal.

It is to be understood that the definitions and explanations of the terms made above apply mutatis mutandis for all aspects described in this specification in the following except as otherwise indicated.

The present invention is based on the finding that the biological persistence, biological half life, biological activity and/or immunogenicity of a neurotoxin can be altered by structurally modifying the neurotoxin light chain. In other words, a modified neurotoxin polypeptide comprising a modified neurotoxin light chain with an altered biological persistence, biological half life, biological activity and/or immunogenicity can be formed from a neurotoxin containing or including a structural modification. Preferably, the duration of biological activity of a neurotoxin can be altered by structurally modifying the neurotoxin light chain as described herein.

In one aspect of the polynucleotide of the invention, said modification confers an increased cleavage by calpain proteases compared to a non-modified light chain.

The term “increased cleavage” as used herein denotes that the cleavage of the modified neurotoxin light chain by calpains is at least 1.5, 2, 3, 4, 5, 10, 50, 100 fold or even higher, in comparison to the cleavage by calpains of a non-modified light chain. The cleavage can be tested by assays well described in the art, for example by ELISA assays, SDS-PAGE, Western blot analysis, and/or HPLC (, e.g. size exclusion). An increased cleavage by calpain proteases of the modified neurotoxin polypeptide comprising a modified neurotoxin light chain described herein results in an increased degradation of the modified neurotoxin in the target cell, e.g. a neuron.

Advantageously, it has been found in accordance with the present invention that the biological persistence, biological half life, biological activity and/or immunogenicity of a neurotoxin can be altered by structurally modifying the light chain of the neurotoxin. The biological persistence and biological activity of BoNT are mainly dependent on the presence of the proteolytically active neurotoxin light chain in the cytoplasm of the target cell, i.e. the neuron. SNARE proteins are cleaved and thereby inactivated by the neurotoxin light chain in sufficient amount only if enough BoNT light chain is present within the neuron. The inactivation of the SNARE proteins, e.g. SNAP-25, within the neuron by the neurotoxin light chain inhibits the exocytosis of neurotransmitter such as acetylcholine. By inhibiting acetylcholine release, the toxin interferes with nerve impulses and causes flaccid (sagging) paralysis of muscles. Thus, the degree of paralysis depends on the concentration of the neurotoxin light chain in the neuron. Over time, the concentration of the neurotoxin light chain is decreased by cellular degradation processes thereby abolishing the blocking effect of the neurotoxin. For example, for BoNT/A the biological persistence is about three months, whereas for BoNT/E it is about four to six weeks. The cellular degradation of neurotoxins is performed by the ubiquitin proteasome system and the calpains. Whereas the light chain of BoNT/E is ubiquitinated and deagraded by the proteasomes the light chain of BoNT/A is resistant against a stable ubiquination and endsuing degradation by the proteasomes. This finding explains as to why BoNT/E is degraded relatively fast in the neuron, resulting in shorter biological persistence and/or duration of biological activity, whereas BoNT/A exhibits a longer biological persistence and/or duration of biological activity. In the recovery process the light chain seems to be continuously degraded by the proteolytic activity of the calpains. Based on these observations, structural modifications have been introduced into the neurotoxin light chains which alter the biological persistence, biological half life, biological activity and/or immunogenicity of the thus modified neurotoxins. Thereby, the neurotoxin can be optimized for the respective disorders to be treated.

In a further aspect of the polynucleotide of the invention, said modification is at least one calpain cleavage site which has been introduced into the light chain.

In this aspect, one, two, three, four, five or even more calpain recognition and/or cleavage site(s) have been introduced into the light chain of neurotoxins in order to increase the degradation of said neurotoxins by calpains, thereby reducing the biological persistence, biological half life, biological activity and/or immunogenicity of the neurotoxin. Advantageously, a thus modified neurotoxin can be used as a medicament for the treatment or prevention of diseases defined herein below. In these indications it is particularly beneficial to use a neurotoxin with a reduced biological persistence, biological half life, biological activity and/or immunogenicity.

According to the literature, no specific amino acid sequence is uniquely recognized by calpains. Amongst protein substrates, primary amino acid sequences and tertiary structure elements seem to be responsible for directing cleavage to a specific substrate. Amongst peptide and small-molecule substrates, the most consistently reported specificity is for small, hydrophobic amino acids (e.g. leucine, valine and isoleucine) at the P2 position, and large hydrophobic amino acids (e.g. phenylalanine and tyrosine) at the P1 position; see e.g. Tompa 2004, J Biol Chem 279, 20775 or Cuerrier 2005, J Biol Chem 280, 40632.

In an aspect, the calpain cleavage site to be introduced into the neurotoxin light chain can be an endogenous calpain recognition and/or cleavage site, i.e. a calpain recognition and/or cleavage site naturally occurring in the neurotoxin light chain. In another aspect, it can be an exogenous calpain recognition and/or cleavage site, i.e. a calpain recognition and/or cleavage site which does not naturally occur in the neurotoxin light chain. Said exogenous calpain recognition and/or cleavage site can also be understood as a heterologous calpain recognition and/or cleavage site or a recombinant calpain recognition and/or cleavage site well understood in the art and as defined herein. Preferably, the calpain recognition and/or cleavage site is a μ-calpain (calpain-1) or m-calpain (calpain-2) recognition and/or cleavage site. More preferably, it is a calpain cleavage site as described in Tompa 2004, J Biol Chem 279, 20775. It is also encompassed by the present invention that the calpain recognition and/or cleavage site comprises the amino acid sequence Pro-Leu-Lys-Ser-Pro-Pro [SEQ ID NO. 13]. In one aspect, said calpain recognition and/or cleavage site replaces the amino acid sequence Ile-Lys-Phe-Ser-Asn-Gly (IKFSNG) [SEQ ID NO. 14] in the BoNT/E light chain, with the isoleucine corresponding to amino acid residue 134, of the BoNT/E light chain as shown, e.g. in GenBank accession number CAA44558.1 or SEQ ID NO: 3.

The introduction of the at least one calpain recognition and/or cleavage site can be carried out by methods described in the art and include mutagenesis techniques as well as standard cloning and PCR based techniques. Recognition and cleavage of the neurotoxin light chain by calpains results in the degradation of the modified neurotoxin light chain. Thereby, the biological persistence, biological half life, biological activity and/or immunogenicity of the neurotoxin is being altered, i.e. decreased, by the incorporation of one or more calpain recognition and/or cleavage site(s) into the neurotoxin light chain, in comparison to a non-modified neurotoxin light chain. This aspect of the invention has been exemplified for BoNT/E.

In still another aspect of the polynucleotide of the invention, said modified light chain and the heavy chain are derived from BoNT/A, BoNT/B, BoNT/C1, BoNT/D, BoNT/F, or BoNT/G.

In this aspect, the light chain (prior to the modification) and the heavy chain come from BoNT/A, BoNT/B, BoNT/C1, BoNT/D, BoNT/F, or BoNT/G. The heavy chain is preferably a non-modified heavy chain, i.e. a naturally-occurring heavy chain. However, it is envisaged that the heavy chain comprises for instance tags which allow for the purification of the modified neurotoxin polypeptide, such as His tag or the like. It is preferred that a protease cleavage site is being introduced between the heavy chain and light chain which allows for proteolytic activation of the modified light chain upon cleavage, e.g. by thrombin or an E. coli protease known in the art.

In an aspect of the polynucleotide of the invention, the modified neurotoxin polypeptide comprising the modified light chain exhibits at least one of the following properties, compared to a non-modified neurotoxin polypeptide: (i) altered, i.e. increased or decreased, half-life time in a cellular system, (ii) altered, i.e. increased or decreased biological persistence, and/or (iii) reduced immunogenicity in an organism, preferably a mammal, more preferably a human. It is preferred that the duration of biological activity is altered, i.e. shortened or prolonged. It is envisaged that in some aspects, the biological activity is also being altered, i.e. increased or decreased.

In a further aspect of the polynucleotide of the invention, said modified neurotoxin polypeptide exhibits a shortened duration of biological activity.

The term “shortened duration of biological activity” as used herein refers to a reduced time period in which the modified neurotoxin light chain exerts its biological activity, in comparison to a non-modified neurotoxin light chain.

Assays for testing the biological activity and the duration of biological activity of neurotoxins are described in the art and comprise, e.g. the Digit Abduction Scoring (DAS) (Aoki 2001, Toxicon 39, 1815) or the voluntary running assay (Keller 2006, Neuroscience 139, 629) or assays described in the following examples. The incorporation of at least one calpain recognition and/or cleavage site into the neurotoxin light chain, results in increased degradation of the neurotoxin light chain, thereby shortening the duration of biological activity. It will be understood that the modified neurotoxin polypeptides according to the present invention will, in an aspect, have reduced side effects when applied to an organism.

In still another aspect of the polynucleotide of the invention, said modification confers a decreased cleavage by calpain proteases compared to a non-modified light chain. The term “said modification confers a decreased cleavage by calpain proteases compared to a non-modified light chain” as used herein means a modification within the neurotoxin light chain which results in decreased cleavage or no cleavage by calpains of the modified light chain, in comparison to a non-modified light chain. Thereby, the degradation of the modified neurotoxin by calpains occurs slower compared to a non-modified neurotoxin, or not at all.

More specifically, the term “decreased cleavage” as used herein denotes that the cleavage of the modified neurotoxin light chain by calpains is at least 1.5, 2, 3, 4, 5 fold or even lower, in comparison to the cleavage by calpains of a non-modified light chain, or no cleavage at all. Assays for testing decreased cleavage by calpain proteases have been mentioned elsewhere herein.

Advantageously, it has been found by the present inventors, that the calpain cleavage site(s) within the light chain of neurotoxins can be mutated, in order to decrease the degradation of said neurotoxins, thereby extending the biological persistence and increasing the biological activity and/or half life. The mutation in an aspect is preferably within an endogenous calpain recognition and/or cleavage site in the neurotoxin light chain. However, it is also envisaged that the mutation is within an exogenous (or heterologous or recombinant) calpain recognition and/or cleavage site in the neurotoxin light chain. A mutation in a nucleic acid sequence as used herein can be a deletion, addition or substitution of one or more nucleotides in the DNA sequence of a calpain recognition and/or cleavage site of a neurotoxin light chain. A mutation in a protein sequence of a calpain recognition and/or cleavage site can be a deletion, addition or substitution of one or more amino acid residues in said protein sequence. As a result of such a mutation, the neurotoxin light chain is no longer recognized, cleaved and thereby degraded by calpains. Mutations to a protein sequence can be the result of mutations to DNA sequences that when transcribed and the resulting mRNA translated produce the mutated protein sequence. In another aspect, the mutation is a deletion of one or more of the endogenous calpain recognition and/or cleavage site(s) within the neurotoxin light chain. In this aspect, the complete endogenous calpain recognition and/or cleavage site(s) within the neurotoxin light chain is/are deleted which results in a neurotoxin light chain no longer being degraded by calpains. One of the reasons, that BoNT/A has originally been selected over the other serotypes, i.e. serotypes BoNT/B, BoNT/C1, BoNT/D, Bo/NT/E, BoNT/F and BoNT/G, for clinical use is that BoNT/A has a substantially longer lasting therapeutic effect, i.e. the inhibitory effect of BoNT/A is more persistent. However, for effective therapy of some medical indications such as, e.g., the treatment of dystonia or for cosmetic purposes it is beneficial to use a BoNT/A with a prolonged persistence, increased biological half life and/or biological activity so that the therapeutic effect can be improved and/or extended. Advantageously, a thus modified neurotoxin can be used as a medicament for the treatment or prevention of diseases specified herein below.

In an aspect of the polynucleotide of the invention, said modification is at least one substitution within a calpain cleavage site in the light chain.

The term “substitution” as used herein means a mutation in which one or more nucleotides within the encoded calpain recognition and/or cleavage site(s) in the neurotoxin light chain is/are replaced by other nucleotides. A substitution in a protein sequence can be a replacement of one or more amino acid residues in the amino acid sequence of the calpain cleavage site. Such a mutation in a protein sequence can be, for example, a substitution of one or more amino acid residues at positions P1, P2, P3, P4, P5, P1′, P2′, and/or P3′ position of the calpain recognition and/or cleavage site in the neurotoxin light chain. One, two, three, four, five, six, seven or even all of the eight amino acid residues can be replaced by another amino acid residue. Preferably, the calpain recognition and/or cleavage site is a μ-calpain (calpain-1) or m-calpain (calpain-2) recognition and/or cleavage site. Mutations to a protein sequence can be the result of mutations to DNA sequences that when transcribed and the resulting mRNA translated produce the mutated protein sequence. Suitable techniques for carrying out such modifications are well known in the art and include standard cloning, mutagenesis techniques as well as PCR based techniques.

In still another aspect of the polynucleotide of the invention, said substitution is a substitution at the P1 or P1′ position of the calpain cleavage site.

In this aspect, position P1 of the calpain recognition and/or cleavage site in the neurotoxin light chain can be substituted e.g. with Valine or Isoleucine.

The P2 position of the calpain recognition and/or cleavage site in the neurotoxin light chain can be replaced with e.g. Tryptophan or Alanine.

The P3 position of the calpain recognition and/or cleavage site in the neurotoxin light chain can be replaced with e.g. Tyrosine or Alanine.

The P4 position of the calpain recognition and/or cleavage site in the neurotoxin light chain can be replaced with e.g. Tryptophan or Phenylalanine.

The P1′ position of the calpain recognition and/or cleavage site in the neurotoxin light chain can be replaced with e.g. Tyrosine or Tryptophan.

The P2′ position of the calpain recognition and/or cleavage site in the neurotoxin light chain can be replaced with e.g. Leucine or Isoleucine.

The P3′ position of the calpain recognition and/or cleavage site in the neurotoxin light chain can be replaced with e.g. Glutamine or Asparagine.

This approach has been exemplified by a modified light chain of BoNT/A in which, inter alia, the P1 position Phenylalanine (corresponding to amino acid residue 213) has been mutated to an Isoleucine residue and the P1′ position Alanine (corresponding to amino acid residue 214) has been mutated to a Tyrosine residue. This modification results in a reduced degradation of the modified neurotoxin light chain by calpains which leads to an extended inhibition of the exocytosis of neurotransmitter, e.g. acetylcholine, thereby increasing the biological persistence, half life and/or biological activity of the neurotoxin.

In an aspect of the polynucleotide of the invention, said modified light chain and the heavy chain are derived from BoNT/A, BoNT/B or BoNT/C.

In a further aspect of the polynucleotide of the invention, said modified neurotoxin polypeptide exhibits a prolonged duration of biological activity.

The term “prolonged duration of biological activity” as used herein refers to a prolonged time period in which the modified neurotoxin light chain exerts its biological activity, in comparison to a non-modified neurotoxin light chain. This can be tested as set forth elsewhere herein.

The present invention also relates to a vector comprising the polynucleotide of the invention. In an aspect, the said vector is an expression vector. The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, in an aspect, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. Moreover, in an aspect of the invention, the polynucleotide is operatively linked to expression control sequences allowing expression in prokaryotic or eukaryotic host cells or isolated fractions thereof in the said vector. Expression of the polynucleotide comprises transcription of the polynucleotide into a translatable mRNA. Regulatory elements ensuring expression in host cells are well known in the art. In an aspect, they comprise regulatory sequences ensuring initiation of transcription and/or poly-A signals ensuring termination of transcription and stabilization of the transcript. Additional regulatory elements may include transcriptional as well as translational enhancers. Possible regulatory elements permitting expression in prokaryotic host cells comprise, e.g., the lac-, trp- or tac-promoter in E. coli, and examples for regulatory elements permitting expression in eukaryotic host cells are the AOX1- or the GAL1-promoter in yeast or the CMV-, SV40-, RSV-promoter (Rous sarcoma virus), CMV-enhancer, SV40-enhancer or a globin intron in mammalian and other animal cells. Other expression systems envisaged by the invention shall permit expression in insect cells, such as polyhedrin promoter based systems.

Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (Invitrogen) or pSPORT1 (Invitrogen) or baculovirus-derived vectors. Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The present invention further relates to a host cell comprising the polynucleotide or the vector of the invention.

The term “host cell” as used herein encompasses prokaryotic and eukaryotic host cells. In an aspect the host cell is a bacterial cell and, in another aspect, a Firmicutes bacterial cell. In one aspect, the said bacterial host cell is an E. coli host cell. In another aspect, it is a Clostridium host cell. In a further aspect, the said Clostridium host cell is a Clostridium botulinum host cell, in even a further aspect, a cell of one of the aforementioned seven different serotypes of Clostridium botulinum. In yet another aspect, the bacterial host cell is a Clostridium tetani host cell. In a further aspect, the host cell is a Bacillus host cell and in a particular aspect a Bacillus megaterium host cell. A eukaryotic host cell, in an aspect, is a cell of an animal cell line suitable for production of toxic proteins or a fungal host cell such as a yeast host cell. A host cell as referred to herein, thus, encompasses in an aspect yeast, mammalian, plant or insect cells either as primary cells or as cell lines.

The present invention also relates to a polypeptide encoded by the polynucleotide of the invention.

The term “polypeptide” as used herein denotes a modified neurotoxin polypeptide comprising a modified neurotoxin light chain and a heavy chain, said modified light chain having at least one modification conferring altered cleavage by calpain proteases, as defined elsewhere herein.

In another aspect, the said light and heavy chain of the neurotoxin polypeptide (prior to the modification of the invention) is a variant having one or more amino acid substitutions, deletions and/or additions. Moreover, such a variant polypeptide, in an aspect, is an amino acid sequence being at least 40%, at least 50%, at least 60%, at least 70%, at least 75%, at least 80%, at least 85%, at least 90%, at least 95%, at least 98% or at least 99% identical to the amino acid sequence as shown in any one of SEQ ID NOs: 1, 2, or 3, or in Swiss-Prot: B1INP5.1, or encoded by a polynucleotide being at least 40%, at least 50%, at least 60%, at least 70%, at least 75%, at least 80%, at least 85%, at least 90%, at least 95%, at least 98% or at least 99% identical to the polynucleotide encoding the amino acid sequence as shown in any one of SEQ ID NOs: 1, 2, or 3, or in Swiss-Prot: B1INP5.1. The term “identical” as used herein refers to sequence identity characterized by determining the number of identical amino acids between two nucleic acid sequences or amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an aspect, each of the aforementioned variant polypeptides (prior to the modification) retains one or more and, in another aspect, all of the biological properties of the respective original neurotoxin polypeptide, i.e. the BoNT/A, BoNT/B, BoNT/C1, BoNT/D, BoNT/E, BoNT/F, BoNT/G or Tetanus Neurotoxin (TeNT). Those of skill in the art will appreciate that full biological activity is maintained only after proteolytic activation, even though it is conceivable that the unprocessed precursor can exert some biological functions or be partially active. “Biological properties” as used herein refers to (a) receptor binding, (b) internalization, (c) translocation across the endosomal membrane into the cytosol, and/or (d) endoproteolytic cleavage of proteins involved in synaptic vesicle membrane fusion. In vivo assays for assessing biological activity include the mouse LD50 assay and the ex vivo mouse hemidiaphragm assay as described by Pearce et al. (Pearce 1994, Toxicol Appl Pharmacol 128: 69-77) and Dressler et al. (Dressler 2005, Mov Disord 20:1617-1619, Keller 2006, Neuroscience 139: 629-637). The biological activity is commonly expressed in Mouse Units (MU). As used herein, 1 MU is the amount of neurotoxic component, which kills 50% of a specified mouse population after intraperitoneal injection, i.e. the mouse i.p. LD50. In an aspect, the variant polynucleotides can be modified according to the invention in that the encoded light chain has at least one modification conferring altered cleacage by calpain proteases as defined herein. In this way, the duration of biological activity of the variant polynucleotides can be changed.

The modified neurotoxin polypeptide of the invention, in an aspect, can be manufactured entirely or in part by chemical synthesis or recombinant molecular biology techniques well known for the skilled artisan. In an aspect, such a method of manufacturing the modified neurotoxin polypeptide of the invention comprises (a) culturing the host cell of the present invention described elsewhere herein and (b) obtaining from the said host cell the polypeptide of the present invention. In an aspect of this method, the polypeptide can be obtained by conventional purification techniques from a host cell lys ate including affinity chromatography, ion exchange chromatography, size exclusion chromatography and/or preparative gel electrophoresis. The modified neurotoxin polypeptide of the invention can be analyzed quantitatively or qualitatively by methods described in the art, such as ELISA assays, Western blot analysis, SDS-PAGE, and/or HPLC (Reverse phase, Size exclusion).

The present invention also relates to a composition comprising the polynucleotide, the vector or the polypeptide of the invention, as a medicament.

The term “medicament” as used herein refers, in one aspect, to a pharmaceutical composition containing the polypeptide, polynucleotide or vector of the present invention as pharmaceutical active compound, wherein the pharmaceutical composition may be used for human or non-human therapy of various diseases or disorders in a therapeutically effective dose.

In an aspect, the polypeptide, polynucleotide or vector of the present invention can be present in liquid or lyophilized form. In an aspect, said compound can be present together with glycerol, protein stabilizers (e.g., human serum albumin (HSA)) or non-protein stabilizers.

The medicament is, in one aspect, administered topically. Conventionally used drug administration is administered intra-muscular, subcutaneous (near glands). However, depending on the nature and the mode of action of a compound (i.e. the polypeptide, polynucleotide or vector of the present invention), the medicament may be administered by other routes as well.

The compound is the active ingredient of the composition, and is in one aspect, administered in conventional dosage forms prepared by combining the drug with standard pharmaceutical carriers according to conventional procedures. These procedures may involve mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables.

The carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or non-toxic, non-therapeutic, non-immunogenic stabilizers and the like.

A therapeutically effective dose refers to an amount of the compound to be used in medicament of the present invention which prevents, ameliorates or treats the symptoms accompanying a disease or condition referred to in this specification. Therapeutic efficacy and toxicity of the compound can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

The medicament referred to herein is administered at least once in order to treat or ameliorate or prevent a disease or condition recited in this specification. However, the said medicament may be administered more than one time.

Specific medicaments are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent. The resulting formulations are to be adapted to the mode of administration. Dosage recommendations shall be indicated in the prescribers or users instructions in order to anticipate dose adjustments depending on the considered recipient.

The medicament according to the present invention may in a further aspect of the invention comprise drugs in addition to the polypeptide, polynucleotide or vector of the present invention which are added to the medicament during its formulation. Finally, it is to be understood that the formulation of a medicament takes place under GMP standardized conditions or the like in order to ensure quality, pharmaceutical security, and effectiveness of the medicament.

In addition, the present invention relates to a composition comprising the polynucleotide of the invention or a polypeptide encoded thereby for use as a medicament for or treating and/or preventing a disease selected from the group consisting of: wound healing, immobilisation for bone and tendon fracture treatment, post surgery immobilization, specifically in connection with haemorrhoidectomy, introduction of dental implants, or hip joint replacement (endoprothesis), knee arthroplasty, ophthalmological surgery, acne, irritable bowel disease or prostate hyperplasia. In an aspect of the composition of the invention, the polynucleotide encodes a modified neurotoxin polypeptide comprising a modified neurotoxin light chain and a heavy chain, said modified light chain having at least one modification conferring altered cleavage by calpain proteases. In one aspect of the composition of the invention, the polynucleotide of the invention or polypeptide encoded thereby comprise a modification conferring an increased cleavage by calpain proteases, compared to a non-modified neurotoxin light chain. In another aspect of the composition of the invention, the modification is at least one calpain cleavage site which has been introduced into the light chain. In a further aspect of the composition of the invention, the modified light chain and the heavy chain are derived from BoNT/A, BoNT/B, BoNT/C1, BoNT/D, BoNT/F, or BoNT/G. In still another aspect of the composition of the invention, the modified neurotoxin polypeptide exhibits a shortened duration of biological activity.

The present invention furthermore relates to a composition comprising the polynucleotide of the invention or a polypeptide encoded thereby for use as a medicament for or treating and/or preventing a disease selected from the group consisting of: voluntary muscle strength, focal dystonia, including cervical, cranial dystonia, and benign essential blepharospasm, hemifacial spasm, and focal spasticity, gastrointestinal disorders, hyperhidrosis, and cosmetic wrinkle correction, Blepharospasm, oromandibular dystonia, jaw opening type, jaw closing type, bruxism, Meige syndrome, lingual dystonia, apraxia of eyelid, opening cervical dystonia, antecollis, retrocollis, laterocollis, torticollis, pharyngeal dystonia, laryngeal dystonia, spasmodic dysphonia/adductor type, spasmodic dysphonia/abductor type, spasmodic dyspnea, limb dystonia, arm dystonia, task specific dystonia, writer's cramp, musician's cramps, golfer's cramp, leg dystonia, thigh adduction, thigh abduction knee flexion, knee extension, ankle flexion, ankle extension, equinovarus, deformity foot dystonia, striatal toe, toe flexion, toe extension, axial dystonia, pisa syndrome, belly dancer dystonia, segmental dystonia, hemidystonia, generalised dystonia, dystonia in lubag, dystonia in corticobasal degeneration, dystonia in lubag, tardive dystonia, dystonia in spinocerebellar ataxia, dystonia in Parkinson's disease, dystonia in Huntington's disease, dystonia in Hallervorden-Spatz disease, dopa-induced dyskinesias/dopa-induced dystonia, tardive dyskinesias/tardive dystonia, paroxysmal dyskinesias/dystonias, kinesiogenic non-kinesiogenic action-induced palatal myoclonus, myoclonus myokymia, rigidity, benign muscle cramps, hereditary chin trembling, paradoxic jaw muscle activity, hemimasticatory spasms, hypertrophic branchial myopathy, maseteric hypertrophy, tibialis anterior hypertrophy, nystagmus, oscillopsia supranuclear gaze palsy, epilepsia, partialis continua, planning of spasmodic torticollis operation, abductor vocal cord paralysis, recalcitant mutational dysphonia, upper oesophageal sphincter dysfunction, vocal fold granuloma, stuttering Gilles de la Tourette syndrome, middle ear myoclonus, protective larynx closure, postlaryngectomy, speech failure, protective ptosis, entropion sphincter Odii dysfunction, pseudoachalasia, nonachalsia, oesophageal motor disorders, vaginismus, postoperative immobilisation tremor, bladder dysfunction, detrusor sphincter dyssynergia, bladder sphincter spasm, hemifacial spasm, reinnervation dyskinesias, cosmetic use craw's feet, frowning facial asymmetries, mentalis dimples, stiff person syndrome, tetanus prostate hyperplasia, adipositas, treatment infantile cerebral palsy strabismus, mixed paralytic concomitant, after retinal detachment surgery, after cataract surgery, in aphakia myositic strabismus, myopathic strabismus, dissociated vertical deviation, as an adjunct to strabismus surgery, esotropia, exotropia, achalasia, anal fissures, exocrine gland hyperactivity, Frey syndrome, Crocodile Tears syndrome, hyperhidrosis, axillar palmar plantar rhinorrhea, relative hypersalivation in stroke, in Parkinsosn's, in amyotrophic lateral sclerosis, spastic conditions, in encephalitis and myelitis autoimmune processes, multiple sclerosis, transverse myelitis, Devic syndrome, viral infections, bacterial infections, parasitic infections, fungal infections, in hereditary spastic paraparesis postapoplectic syndrome hemispheric infarction, brainstem infarction, myelon infarction, in central nervous system trauma, hemispheric lesions, brainstem lesions, myelon lesion, in central nervous system hemorrhage, intracerebral hemorrhage, subarachnoidal hemorrhage, subdural hemorrhage, intraspinal hemorrhage, in neoplasias, hemispheric tumors, brainstem tumors, myelon tumor and vaginism.

In an aspect of the composition of the invention, the polynucleotide encodes a modified neurotoxin polypeptide comprising a modified neurotoxin light chain and a heavy chain, said modified light chain having at least one modification conferring altered cleavage by calpain proteases. In one aspect of the composition of the invention, the polynucleotide of the invention or polypeptide encoded thereby comprise a modification which confers a decreased cleavage by calpain proteases compared to a non-modified light chain. In another aspect of the composition of the invention, the modification is at least one substitution within a calpain cleavage in the light chain. In still another aspect of the composition of the invention, substitution is a substitution at the P1, P2, P3, P4, P5, P1′, P2′, and/or P3′ position of the calpain cleavage site. In a further aspect of the composition of the invention, modified light chain and the heavy chain are derived from BoNT/A, BoNT/B or BoNT/C1. In a still further aspect of the composition of the invention, the modified neurotoxin polypeptide exhibits a prolonged duration of biological activity.

Another aspect of the invention, the composition relates to a cosmetic composition which can be formulated as described for a medicament above. For a cosmetic composition, likewise, it is envisaged that the modified neurotoxin of the present invention is in an aspect used in substantially pure form. Cosmetic compositions are, in a further aspect, to be applied intramuscular. In an even further aspect, cosmetic compositions comprising the neurotoxin can be formulated as anti-wrinkle solution.

All references cited in this specification are herewith incorporated by reference with respect to their entire disclosure content and the disclosure content specifically mentioned in this specification.

### EXAMPLES

The invention will now be illustrated by examples which shall, however, not be construed as limiting the scope of the invention.

### Example 1

### Different Susceptibility of BoNT/E and BoNT/A for m-Calpain

30 μg BoNT/E (SEQ ID NO: 3) in 162 mM Na Acetate buffer were supplemented with 14.7 μL 119 mM dithiothreitole to cleave the disulphide bond between heavy and light chain. After incubation at 30° C. for 30 min 90 μL 125 mM HEPES, 375 mM NaCl and 10 mM CaCl₂, pH 7.5 were added followed by 2 μg calpain (Calbiochem) in 3 μL. After 6, 24 and 46 hours incubation 23 μL sample were supplemented with 2 μL EGTA and then analysed by SDS-PAGE. 30 μg of BoNT/A (SEQ ID NO: 1) were analysed in parallel in the same buffer and under the same conditions and analysed by SDS-PAGE. The electropherogram shows that the light chain of BoNT/A was resistant against proteolysis for 46 hours whereas the heavy chain is slightly degraded, a band with a molecular weight of about 80 kD appeared and the intensity of the heavy chain (about 100 kD) was reduced. In contrast the heavy chain of BoNT/E was completely degraded after 360 min. The light chain of BoNT/E was still visible after 48 hours but showed a loss of intensity. After 48 hours the light chain had disappeared i.e. was completely degraded.

### Example 2

### Preparation of a Mutated BoNT/A

The light chain of BoNT/A comprised by SEQ ID NO: 1 contains a putative cleavage site for m-calpain, as shown in SEQ ID NO: 5. The following mutants were synthesised: E126A/L127A and F213I/A214I, with the mentioned mutations at the indicated positions within the BoNT/A light chain comprised by SEQ ID NO: 1. The double mutations were introduced into the expressin plasmid pET29c-mod Strep-BoNTA-Strep-His, containing the BoNT/A gene and the genetic information for the c-terminal Strep- and His-tag and a thrombin cleavage site between heavy and light chain. The introduction was accomplished by site directed mutagenesis according to the Gentailor protocol (Invitrogen).

Plasmids with the verified DNA were transformed into E. coli BL21DE3. Colonies were grown in YT medium+50 μg/mL kanamycin over night at 37° C. 3 L 2YT medium were inoculated with this pre-culture. After reaching an OD 0.6 the expression was started by adding 50 μM IPTG until a final concentration of 0.2 M IPTG was attained. After further growth for 14 hours the cells were harvested. The cell pellets were lysed by ultrasonication in 50 mM Tris, 150 mM NaCl, 1.5 mM imidazole in the presence of complete protease inhibitor cocktail without EDTA (Roche). After centrifugation the mutants were purified over 2 mL Talon, Matrix (Clontech), bound proteins were eluted with 250 mM imidazole. Mutant containing fractions were further purified by cation chromatography HiTrap SP FF in 50 mm Tris/HCl pH 8.0. The mutants were eluted with a NaCl gradient 0-1.0 M NaCl. The purified single chain mutated BoNT/A was cleaved into heavy and light chain with 0.01 U Thrombin per μg protein at ambient temperature overnight. Thrombin was removed by size exclusion chromatography over a HiLoad Superdex 200 16/60 column in phosphate buffered saline at pH 7.4. The biological activity was analysed in the mouse hemidiaphragm assay.

### Example 3

### Analysis of the Duration of Effect in the Mouse Running Assay

Mice were trained for about 14 days in a cage equipped with a running wheel according to Keller (loc. cit.). The running distance per night was monitored electronically. 0.8 Units of unmodified BoNT/A in 20 μl were injected into the gastrocnemius muscle of 8 mice. The running distance was reduced to zero after 3-4 days. Over a period of 28 days the running distance continuously increased to reach the initial value. In a further experiment a group of mice is treated with 0.8 U of the mutant BoNT/A generated in Example 2 in parallel with unmodified BoNT/A. Again after 3-4 days the running distance is reduced to zero and continuously increases for both groups. The recovery time for mice treated with the mutant BoNT/A is markedly longer.

1-16. (canceled)

17. A polynucleotide encoding a modified neurotoxin polypeptide comprising a modified neurotoxin light chain and a heavy chain, wherein the modified neurotoxin light chain exhibits at least one modification conferring altered cleavage by calpain proteases.

18. The polynucleotide of claim 17, wherein the at least one modification confers an increased cleavage by calpain proteases compared to a non-modified neurotoxin light chain.

19. The polynucleotide of claim 18, wherein the at least one modification is at least one calpain cleavage site which has been introduced into the neurotoxin light chain.

20. The polynucleotide of claim 19, wherein the modified neurotoxin light chain and the heavy chain are derived from BoNT/A, BoNT/B, BoNT/C1, BoNT/D, BoNT/F, or BoNT/G.

21. The polynucleotide of claim 17, wherein the modified neurotoxin polypeptide exhibits a shortened duration of biological activity.

22. The polynucleotide of claim 17, wherein the at least one modification confers a decreased cleavage by calpain proteases compared to a non-modified neurotoxin light chain.

23. The polynucleotide of claim 22, wherein the at least one modification is at least one substitution within a calpain cleavage site in the neurotoxin light chain.

24. The polynucleotide of claim 23, wherein the at least one substitution is a substitution at the P1, P2, P3, P4, P5, P1′, P2′, and/or P3′ position of the calpain cleavage site.

25. The polynucleotide of claim 23, wherein the modified neurotoxin light chain and the heavy chain are derived from BoNT/A, BoNT/B or BoNT/C1.

26. The polynucleotide of claim 22, wherein the modified neurotoxin polypeptide exhibits a prolonged duration of biological activity.

27. A vector comprising the polynucleotide of claim 17.

28. A host cell comprising the polynucleotide of claim 17.

29. A host cell comprising the vector of claim 27.

30. A polypeptide encoded by the polynucleotide of claim 17.

31. A pharmaceutical composition comprising the polypeptide of claim 30.

32. A method for treating and/or preventing a disease or disorder comprising administering to a subject in need thereof a therapeutically effective dose of a composition comprising the polynucleotide of claim 17 or a polypeptide encoded thereby.

33. The method of claim 32, wherein the disease or disorder is selected from the group consisting of: wound healing, immobilisation for bone and tendon fracture treatment, post surgery immobilization, post surgery immobilization after haemorrhoidectomy, post surgery immobilization after introduction of dental implants, post surgery immobilization after hip joint replacement (endoprothesis), post surgery immobilization after knee arthroplasty, post surgery immobilization after ophthalmological surgery, acne, irritable bowel disease and prostate hyperplasia.

34. A method for treating and/or preventing a disease or disorder comprising administering to a subject in need thereof a therapeutically effective dose of a composition comprising the polynucleotide of claim 22 or a polypeptide encoded thereby.

35. The method of claim 34, wherein the disease or disorder is selected from the group consisting of: voluntary muscle strength, focal dystonia, cervical dystonia, cranial dystonia, benign essential blepharospasm, hemifacial spasm, focal spasticity, gastrointestinal disorders, hyperhidrosis, cosmetic wrinkle correction, blepharospasm, oromandibular dystonia jaw opening type, oromandibular dystonia jaw closing type, bruxism, Meige syndrome, lingual dystonia, apraxia of eyelid, opening cervical dystonia, antecollis, retrocollis, laterocollis, torticollis, pharyngeal dystonia, laryngeal dystonia, spasmodic dysphonia/adductor type, spasmodic dysphonia/abductor type, spasmodic dyspnea, limb dystonia, arm dystonia, task specific dystonia, writer's cramp, musician's cramps, golfer's cramp, leg dystonia, thigh adduction, thigh abduction knee flexion, knee extension, ankle flexion, ankle extension, equinovarus, deformity foot dystonia, striatal toe, toe flexion, toe extension, axial dystonia, pisa syndrome, belly dancer dystonia, segmental dystonia, hemidystonia, generalised dystonia. dystonia in corticobasal degeneration, dystonia in lubag, tardive dystonia, dystonia in spinocerebellar ataxia, dystonia in Parkinson's disease, dystonia in Huntington's disease, dystonia in Hallervorden-Spatz disease, dopa-induced dyskinesias/dopa-induced dystonia, tardive dyskinesias/tardive dystonia, paroxysmal dyskinesias/dystonias, kinesiogenic non-kinesiogenic action-induced palatal myoclonus, myoclonus myokymia, rigidity, benign muscle cramps, hereditary chin trembling, paradoxic jaw muscle activity, hemimasticatory spasms, hypertrophic branchial myopathy, maseteric hypertrophy, tibialis anterior hypertrophy, nystagmus, oscillopsia supranuclear gaze palsy, epilepsia, partialis continua, planning of spasmodic torticollis operation, abductor vocal cord paralysis, recalcitrant mutational dysphonia, upper oesophageal sphincter dysfunction, vocal fold granuloma, stuttering, Gilles de la Tourette syndrome, middle ear myoclonus, protective larynx closure, postlaryngectomy, speech failure, protective ptosis, entropion sphincter Odii dysfunction, pseudoachalasia, nonachalsia, oesophageal motor disorders, vaginismus, postoperative immobilisation tremor, bladder dysfunction, detrusor sphincter dyssynergia, bladder sphincter spasm, hemifacial spasm, reinnervation dyskinesias, crow's feet, frowning facial asymmetries, mentalis dimples, stiff person syndrome, tetanus prostate hyperplasia, adipositas, treatment infantile cerebral palsy strabismus, mixed paralytic concomitant, after retinal detachment surgery, after cataract surgery, aphakia myositic strabismus, myopathic strabismus. dissociated vertical deviation, strabismus surgery, esotropia, exotropia, achalasia, anal fissures, exocrine gland hyperactivity, Frey syndrome, Crocodile Tears syndrome, hyperhidrosis, axillar palmar plantar rhinorrhea, relative hypersalivation in stroke, Parkinson's Disease, amyotrophic lateral sclerosis, spastic conditions, encephalitis and myelitis autoimmune processes, multiple sclerosis, transverse myelitis, Devic syndrome, viral infections, bacterial infections, parasitic infections, fungal infections, hereditary spastic paraparesis, postapoplectic syndrome hemispheric infarction, brainstem infarction, myelon infarction, central nervous system trauma, hemispheric lesions, brainstem lesions, myelon lesion, central nervous system haemorrhage, intracerebral hemorrhage, subarachnoidal hemorrhage, subdural hemorrhage, intraspinal hemorrhage, neoplasias, hemispheric tumors, brainstem tumors, myelon tumor and vaginism.


SEQUENCE LISTING (14 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 3 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 4 - protein, 7 aa, Artificial
  Calplain recognition and/or cleavage site within BoNT/A
GKFATDP

SEQ ID NO: 5 - protein, 6 aa, Artificial
  Calpain recognition and/or cleavage site within BoNT/A
ELKVID

SEQ ID NO: 6 - protein, 6 aa, Artificial
  Calpain recognition and/or cleavage site within BoNT/A
EDTSGK

SEQ ID NO: 7 - protein, 6 aa, Artificial
  Calpain recognition and/or cleavage site within BoNT/A
GLEVSF

SEQ ID NO: 8 - protein, 6 aa, Artificial
  Calpain recognition and/or cleavage site within BoNT/A
LNKAKS

SEQ ID NO: 9 - protein, 6 aa, Artificial
  Calpain recognition and/or cleavage site within BoNT/A
VDKLKF

SEQ ID NO: 10 - protein, 6 aa, Artificial
  Calpain recognition and/or cleavage site within BoNT/A
VLNRKT

SEQ ID NO: 11 - protein, 6 aa, Artificial
  Calpain recognition and/or cleavage site within BoNT/A
IVGTTA

SEQ ID NO: 12 - protein, 6 aa, Artificial
  Calpain recognition and/or cleavage site within BoNT/A
GTTASL

SEQ ID NO: 13 - protein, 6 aa, Artificial
  Calpain recognition and/or cleavage site
PLKSPP

SEQ ID NO: 14 - protein, 6 aa, Artificial
  Calpain recognition and/or cleavage site within BoNT/E
IKFSNG